(19) United States
(12) Patent Application Publication
(10) Pub. No.: US 2023/0270402 A1
(43) Pub. Date: Aug. 31, 2023

(54) IMAGE BASED LUNG AUSCULTATION SYSTEM AND METHOD FOR DIAGNOSIS

(71) Applicant: HILL-ROM SERVICES PTE. LTD., Singapore (SG)

(72) Inventors: Yaolong LOU, Singapore (SG); Susan E. MALARET, Chanhassen, MN (US); Stacey REULAND, St. Paul, MN (US); Eng Keong TAY, Singapore (SG); Chang Sheng LEE, Punggol (SG); Karen MULLERY, St. Paul, MN (US)

(21) Appl. No.: 18/168,654

(22) Filed: Feb. 14, 2023

Related U.S. Application Data
(60) Provisional application No. 63/314,499, filed on Feb. 28, 2022.

(51) Int. Cl. A61B 7/00; A61B 7/04

(52) U.S. Cl. CPC A61B 7/003; A61B 7/04

(57) ABSTRACT

An image based lung auscultation system may include an acoustic sensing unit and a data capture and processing unit. The acoustic sensing unit may be positionable on a patient. The data capture and processing unit may include a camera, a user interface, and a controller.

### CROSS-REFERENCE TO RELATED APPLICATIONS

This application claims priority under 35 U.S.C. § 119(e) to U.S. Provisional Patent Application No. 63/314,499, filed Feb. 28, 2022, which is expressly incorporated by reference herein.

### BACKGROUND

The present disclosure relates to an image based lung auscultation system used to diagnose lung diseases. More specifically, the present disclosure is related to a lung auscultation system providing images can be used by a doctor or medical professional to diagnose lung diseases.

The most direct way to access lung health condition is to visualize the lung by imaging. Lung health is usually provided by chest images from x-ray, computed tomography (CT), and magnetic resonance imaging (MRI) techniques. These techniques are suitable for visualizing the airways and lung pathology. However, the cumbersome and unwieldy equipment required to prepare these images require that the images be captured at the equipment and must not be impeded by foreign objects such as clothing, jewelry, or the like. Electrical impedance tomography (EIT) is an imaging technology that can be implemented to provide portability to patients, but still requires the removal of clothing and the like to apply electrodes on the patient's skin on their chest and back. Vibration response imaging (VRI) by acoustic signals is another technique that is portable to the patient, but also suffers the drawback of attaching multiple sensors to the patient's skin.

Auscultation has been a known traditional method in lung diagnosis. Auscultation is a listening method which has been used with a traditional stethoscope and which depends on aural comprehension, analysis, and experience by a doctor. However, auscultation lacks the immediate visualization or imaging of a patient's lungs to assist the doctor with diagnosis. Instead, lung diagnostic imaging is achieved with X-ray, MRI, CT, and/or other stationary, large, and heavy machinery. These methods of lung diagnostic imaging are not typically readily available to primary care providers and/or pulmonology clinics, which may be time consuming and/or may result in significant costs for the patient and/or doctor. Moreover, X-ray and CT expose patients to radiation, which may be harmful or impractical for some patients. An equipment-to-patient method, such as VRI, may cause discomfort and/or may be time consuming due to attaching multiple sensors to the patient's skin.

Thus, there exists a need for a diagnostic tool that generates an image of a patient's lungs while the doctor performs auscultation on the patient, improves patient comfort, minimizes time and costs spent on lung diagnostic imaging, and provides an efficient diagnostic tool for the doctor.

### SUMMARY

The present disclosure includes one or more of the features recited in the appended claims and/or the following features which, alone or in any combination, may comprise patentable subject matter.

In a first aspect of the disclosed embodiments, an image based lung auscultation system may include an acoustic sensing unit and a data capture and processing unit. The acoustic sensing unit may be positionable on a patient. The acoustic sensing unit may also include a controller and an acoustic sensor to capture and communicate an acoustic signal from a respiratory cycle of the patient. The data capture and processing unit may include a camera, a user interface, and a controller. The camera may be operable by a user to position the acoustic sensing unit on the patient. The user interface may include user inputs, a display, and a processor and a memory device storing instructions that, when executed by the processor, receive the user inputs, display an image generated by the camera on the display, display real-time information of the acoustic signal on the display, and display an output representing the patient's lungs on the display. The user inputs may operate the data capture and processing unit. The controller may include a processor and a memory device storing instructions that, when executed by the processor, receive and store the acoustic signal from the acoustic sensing unit, generate real-time information of the acoustic signal, generate the output of the patient's lung(s), and communicate the real-time information and the output to the user interface.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is an image based lung auscultation system of the present disclosure, the system including an acoustic sensing unit embodied as an electronic stethoscope shown positioned on a patient and connected to a data capture and processing unit embodied as a mobile display device, the system operable to capture and display information indicative of the function of the patient's lungs;

FIG. 2 is a block diagram of the system of FIG. 1;

FIG. 3 is a view of a display of a user interface during use of the system;

FIG. 4 is a view of the display of the user interface displaying information detected by the system;

FIG. 5 is a view of the display showing a set of dynamic grayscale acoustic images representing the acoustic signals from the patient's lungs over a given time interval;

FIG. 6 is a first representation of the display showing a dynamic grayscale acoustic image representing the patient's lung function after the acoustic signals have been processed by the data capture and processing unit;

FIG. 7 is a second representation of the display showing a second dynamic grayscale acoustic image representing the patient's lung function after the acoustic signals have been processed by the data capture and processing unit; and

FIG. 8 is a flow chart illustrating the process of capturing and converting the acoustic signals captured from the patient's lungs to an image representing the patient's lungs.

### DETAILED DESCRIPTION

Auscultation has been a traditional method in lung diagnosis. Auscultation is a listening method which has been used with a traditional stethoscope and which depends on aural comprehension, analysis, and experience by a doctor. However, auscultation lacks the immediate visualization or imaging of a patient's lungs to assist the doctor with diagnosis.

The current disclosure describes an image based lung auscultation system 10 and a method for implementing the image based lung auscultation system 10 to facilitate auscultation. The image based lung auscultation system 10 utilizes information obtained from a respiratory sound signal (acoustic signal) and provides an image 58, 64, 66 representing a patient' lung(s). As will be described below, the image based lung auscultation system 10 processes the acoustic signal(s) from at least one respiratory cycle of the patient's lung(s) and converts the acoustic signals into an image 58, 64, 66 representing the lung function of the patient 16. In this context, the “image” 58, 64, 66 is a digital construction, which may be visualized graphically, based on the intensity of sounds emanating from the lungs of the patient 16. The image 58, 64, 66 may be at least one dynamic grayscale acoustic image.

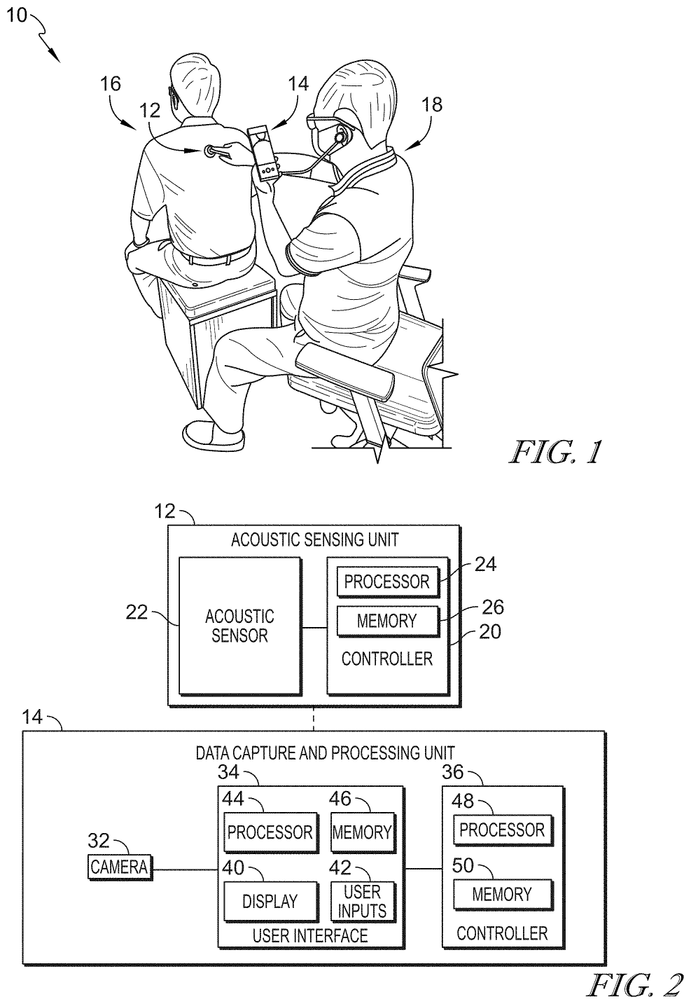

In the illustrative embodiment of FIGS. 1 and 2, the image based lung auscultation system 10 is shown. The image based lung auscultation system 10 is used on a patient 16 by a user 18. The image based lung auscultation system 10 includes an acoustic sensing unit 12 embodied as an electronic stethoscope 12 and a data capture and processing unit 14 embodied as a mobile display device, phone and/or tablet 14. The acoustic sensing unit 12 is positioned on a patient's back by the user 18 to capture at least one acoustic signal from at least one respiratory cycle. The data capture and processing unit 14 is operable to direct the user 18 to position the acoustic sensing unit 12 on the patient's back, display real-time information 55, 56, 57 during the at least one respiratory cycle, display an image 58, 64, 66 representing the patient's lung(s), and process the at least one acoustic signal from the at least one respiratory cycle. In the present embodiment, the acoustic sensing unit 12 is positioned over the patient's shirt. In other embodiments, the acoustic sensing unit may be in contact with the patient's skin. In some embodiments, the acoustic sensing unit 12 may be held in place on the patient's back by the user 18. In other embodiments, the acoustic sensing unit 12 may be secured to the patient's back, such as with a band, an adhesive, or any other suitable devices or methods capable of performing the functions described herein.

As shown diagrammatically in FIG. 2, the acoustic sensing unit 12 and the data capture and processing unit 14 communicate with each other to capture and process the at least one acoustic signal from the patient's lungs. In some embodiments, the acoustic sensing unit 12 and the data capture and processing unit 14 communicate wirelessly, such as by Bluetooth, Wi-Fi, or any other suitable devices or methods capable of communicating wirelessly. In other embodiments, a wired connection is provided between the acoustic sensing unit 12 and the data capture and processing unit 14, such as USB, Ethernet, or any other suitable devices or methods capable of communicating by wired connection.

The acoustic sensing unit 12 has a controller 20 and an acoustic sensor 22, as shown in FIG. 2. The controller 20 has a processor 24 and a memory device 26 storing instructions that, when executed by the processor 24, cause the acoustic sensor 22 to capture the at least one acoustic signal from the at least one respiratory cycle of the patient 16 and communicate the at least one acoustic signal to the data capture and processing unit 14. The acoustic sensing unit 12 is illustrated as an electronic stethoscope in FIG. 1; however, it will be appreciated that the acoustic sensing unit 12 may be any acoustic sensing unit 12 suitable to collect acoustic signals from a patient's respiratory system and/or suitable to perform the functions described herein. In other embodiments, the acoustic sensing unit 12 may be more than one acoustic sensing unit 12. In further embodiments, the acoustic sensing unit 12 may have more than one acoustic sensor 22. In the present embodiment, the controller 20 and the acoustic sensor 22 are both contained within the acoustic sensing device 12. However, in other embodiments, the controller 20 may be separate from the acoustic sensing device 12 and may be connected to the acoustic sensing device 12 by wireless or wired connection(s) as described above. In further embodiments, the acoustic sensing unit 12 may store the at least one acoustic signal and transfer the at least one acoustic signal to the data capture and processing unit 14 after all data has been collected. This transfer of data may be achieved with wireless or wired connection(s) as described above.

The data capture and processing unit 14 has a camera 32, a user interface 34, and a controller 36, as shown in FIG. 2. The camera 32 is operable by the user 18 to position the acoustic sensing unit 12 on the patient 16, such as the patient's back. The user interface 34 displays information captured and processed by the image based lung auscultation system 10 and receives user inputs 42. The controller 36 receives and processes the at least one acoustic signal from the acoustic sensing unit 12 to be displayed on the user interface 34. The data capture and processing unit 14 is illustrated as a mobile phone and/or tablet 14, however the data capture and processing unit 14 may be any device(s) 14 capable of processing the acoustic signals from the at least one acoustic sensor 12 and/or performing the functions described herein. Such device(s) 14 may include, but are not limited to, a server (e.g., stand-alone, rack-mounted, blade, etc.), a network appliance (e.g., physical or virtual), a high-performance computing device, a web appliance, a distributed computing system, a computer, a processor-based system, a multiprocessor system, a smartphone, a tablet computer, a laptop computer, a notebook computer, a mobile computing device, and any type of computation or computer device.

In the present embodiment, as shown in FIG. 2, the camera 32 is integrated into the data capture and processing unit 14 and is operated by the user 18 via the user interface 34. In other embodiments, the camera 32 may be separate from the data capture and processing unit 14. In such embodiments, the camera 32 may be operated by the user 18 via the user interface 34 or via a button or control on or connected to the camera 32. Additionally, in such embodiments, the camera 32 may be connected to the data capture and processing unit 14 and/or the user interface 34 by wireless or wired connection(s) as described above. In other embodiments, the camera 32 may be any image capturing device 32 capable of performing the functions described herein.

The user interface 34, as shown in FIG. 2, has a display 40 which displays a video or image captured by the camera 32, real-time information 55, 56, 57 of at least one acoustic signal, and/or the image 58, 64, 66 of the patient's lung(s). The user interface 34 also receives user inputs 42 to control what is shown in the display 40. Additionally, the user interface 34 has a processor 44 and a memory device 46 storing instructions that, when executed by the processor 44, receive user inputs 42, operate the camera 32, display the video or image of the patient 16 on the display 40, display real-time information 55, 56, 57 of the acoustic signals on the display 40, and/or display the image 58, 64, 66 of the patient's lung(s) on the display 40.

In the present embodiment, as shown in FIG. 1, the display 40 is integrated into a mobile phone and/or tablet 14. Additionally or alternatively, the display 40 may be or may be integrated into a laptop computer, a large screen monitor, a projection on a wall, or any suitable device or method for the patient 16 and/or the user 18 to view the information on the display 40 and/or perform the functions described herein. In the present embodiment, the display 40 is integrated into the data capture and processing unit 14 and the user interface 34. Additionally or alternatively, the display 40 may be separate from the data capture and processing unit 14 and/or the user interface 34. In such embodiments, the display 40 may be connected to the data capture and processing unit 14 and/or the user interface 34 by wireless or wired connection(s) as described above.

The user inputs 42 operate the camera 32, choose a guide to help the user 18 position the acoustic sensor 12 on the patient 16, and/or select information to be shown on the display 40, as shown in FIG. 2. The user inputs 42 may be received via a touch-screen display 40, as shown in the present embodiment. In other embodiments, the user inputs 42 may be received via a keyboard, a computer mouse, or any suitable devices or methods capable of performing the functions described herein. In such embodiments, the user inputs 42 may be communicated to the data capture and processing unit 14 and/or the user interface 34 by wireless or wired connection(s) as described above.

In the present embodiment, the processor 44 and the memory device 46 are integrated into the data capture and processing unit 14 and the user interface 34, as shown in FIG. 2. In other embodiments, the processor 44 and/or the memory device 46 may be separate from the data capture and processing unit 14 and/or the user interface 34. In such embodiments, the processor 44 and/or the memory device 46 may be connected to the data capture and processing unit 14 and/or the user interface 34 by wireless or wired connection(s) as described above.

The controller 36, as shown in FIG. 2, has a processor 48 and a memory device 50. The processor 48 executes instructions stored on the memory device 50, the instructions including receiving and storing the at least one acoustic signal from the acoustic sensing unit 12, processing the at least one acoustic signal, generating real-time information 55, 56, 57 of the at least one acoustic signal, synchronizing the at least one acoustic signal from at least one respiratory cycle, generating the image 58, 64, 66 of the patient's lung(s), and communicating the real-time information 55, 56, 57 and/or the image 58, 64, 66 to the user interface 34. In other embodiments, the processor 48 may also execute instructions stored on the memory device 50 that implement artificial intelligence to provide a diagnosis based on the at least one acoustic signal, real-time information 55, 56, 57, and/or the image 58, 64, 66. In the present embodiment, the controller 36 is integrated into the data capture and processing unit 14. In other embodiments, the controller 36 may be separate from the data capture and processing unit 14. In such embodiments, the controller 36 may be connected to the data capture and processing unit 14 by wireless or wired connection(s) as described above. In some embodiments, the controller 36 may upload the at least one acoustic signal, real-time information 55, 56, 57, and/or the image 58, 64, 66 to an external server or cloud device by wireless or wired connection(s) to store and/or share with the patient 16, doctor, and/or other medical professionals. In such embodiments, artificial intelligence may be implemented to provide a diagnosis based on the at least one acoustic signal, real-time information 55, 56, 57, and/or the image 58, 64, 66.

In some embodiments, the processor 44 of the user interface 34 and the processor 48 of the controller 36, as shown in FIG. 2, may be one processor. In such embodiments, the one processor may execute instructions stored on memory device 46 and memory device 50. Alternatively or additionally, the memory device 46 and the memory device 50 may be one memory device. In such embodiments, the one memory device may store instructions stored on memory device 46 and memory device 50.

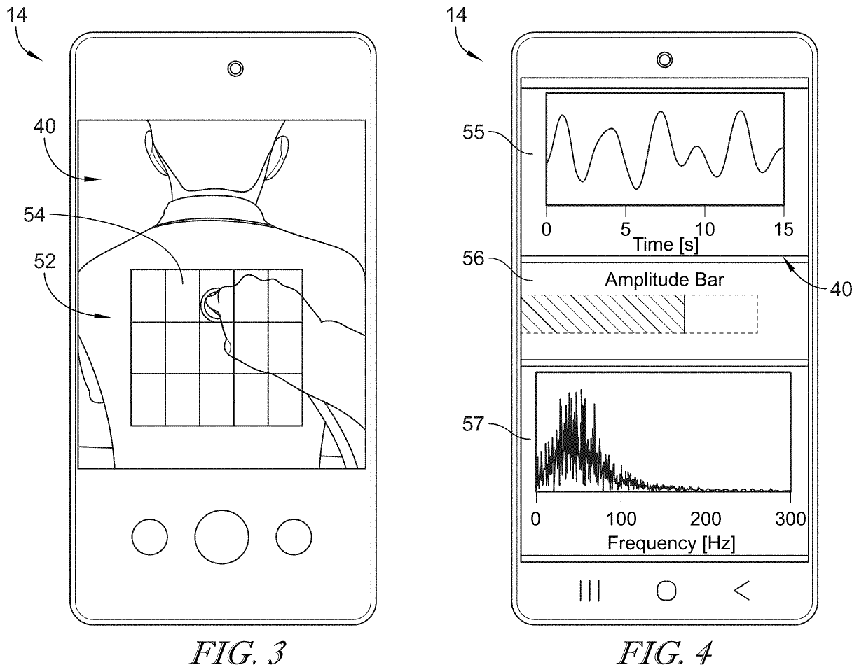

Referring to FIG. 3, the display 40 displays a grid 52 while the user 18 operates the camera 32. The grid 52 guides the user 18 with positioning the acoustic sensing unit 12 on the patient's back. The grid 52 has at least one designated location 54 which is referenced by the user 18 to position the acoustic sensing unit 12 on the patient's back. In some embodiments, the user 18 may be able to adjust the number of designated locations 54 on the grid 52 via user inputs 42. In further embodiments, the grid 52 may not be used and/or the data processing and capture unit 14 may record the position of the acoustic sensing unit 12 on the patient's back without the grid 52.

The display 40 displays real-time information 55, 56, 57 of one acoustic signal of one respiratory cycle while the acoustic signal is captured by the acoustic sensing unit 12, as shown in FIG. 4. In the present embodiment, the real-time information 55, 56, 57 of one acoustic signal that is displayed on the display 40 includes the breathing signal against time 55, the current and max amplitude 56, and the power spectrum against frequency 57. In some embodiments, any combination of any information 55, 56, 57 processed from one acoustic signal of one respiratory cycle may be displayed on the display 40. In other embodiments, information 55, 56, 57 from more than one acoustic signal may be displayed on the display 40. Alternatively or additionally, the user 18 may be able to adjust the real-time information 55, 56, 57 that is displayed on the display 40 via user inputs 42.

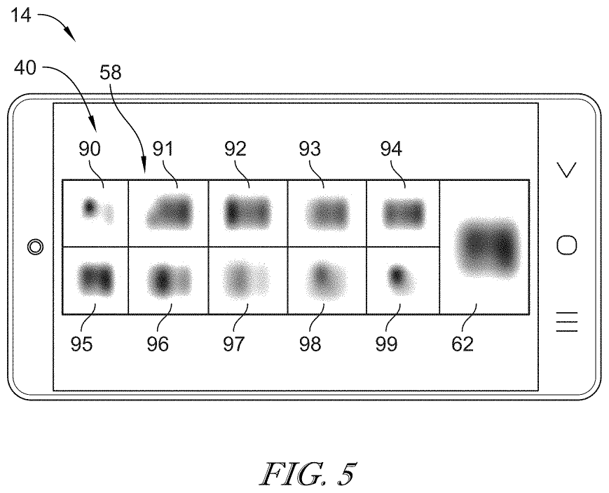

After the acoustic sensing unit 12 and the data capture and processing unit 14 collect at least one acoustic signal, the controller 36 synchronizes the at least one acoustic signal and generates the image 58 to be displayed on the display 40, as shown in FIG. 5. In the present embodiment, the image 58 is at least one dynamic grayscale acoustic image 58 of the patient's lungs over a period of time. The generation of the at least one dynamic grayscale acoustic image 58 is achieved using an interpolation method such as that disclosed as steps 344 and 346 in U.S. Provisional Patent Application No. 63/252,250, which is incorporated by reference herein for the disclosure of estimating the acoustic intensity of acoustic signals to generate a dynamic grayscale acoustic image representing the acoustic signals and/or the patient's lungs.

As shown in FIG. 5, the image 58 includes sequential frames 90, 91, 92, 93, 94, 95, 96, 97, 98, 99 and a simulated acoustic image 62. In the present embodiment, each sequential frame 90-99 includes a dynamic grayscale acoustic image. The dynamic grayscale acoustic image in each sequential frame 90-99 shows the regional acoustic intensity of the patient's lungs during a period of time. In the present embodiment, the top row of sequential frames 90-94 shows an inspiratory phase of one respiratory cycle divided over 0.4 second time periods, the second row of sequential frames 95-99 shows an expiratory phase of one respiratory cycle divided over 0.4 second time periods. Sequential frame 90, as an example, shows the acoustic intensity of the patient's lungs captured at the designated location 54 over a 0.4 second time period at the beginning of one respiratory cycle. Sequential frame 91 shows the acoustic intensity of the patient's lungs captured at another designated location 54 during the next 0.4 second time period within the same respiratory cycle. In other embodiments, the sequential frames 90-99 may be more than or less than ten sequential frames 90-99. In some embodiments, the time period may be more than or less than 0.4 seconds. In further embodiments, more than one respiratory cycle may be represented by the sequential frames 90-99 and/or simulated acoustic image 62. The user 18 may adjust the image 58, such as the number of sequential frames 90-99, on the display 40 with user inputs 42.

In the present embodiment, the simulated acoustic image 62 represents the maximal mean intensity of the dynamic grayscale acoustic images in the ten sequential frames 90-99. In other embodiments, the simulated acoustic image 62 may represent one or more additional features of the dynamic grayscale acoustic images which may assist with diagnosis of the patient's lungs.

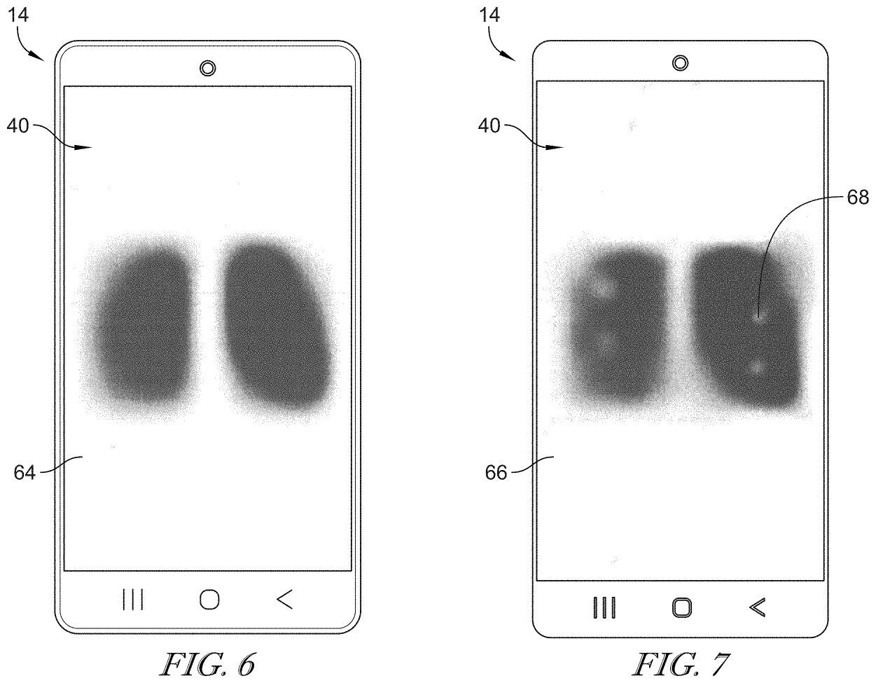

FIGS. 6 and 7 show an image 64, 66 generated by the controller 36 displayed on the display 40. In the present, embodiment, the image 64, 66, is the simulated acoustic image 62 as described above. More specifically, FIG. 6 shows the image 64 of healthy lungs on the display 40. On the other hand, FIG. 7 shows the image 66 of lungs which have obstructed airways 68. In the present embodiment, the obstructed airways 68 are generated in the image 66 by increasing the airway wall thickness of large airways by a mean factor of 2.3 and of small airways by a mean factor of 1.5. In other embodiments, the obstructed airways 68 generated in the image 66 may be achieved by a different mean factor for the large airways and/or small airways, or by another parameter or factor based on the at least one acoustic signal. In some embodiments, the image 64, 66 may be a dynamic grayscale acoustic image generated by other methods, calculations, or parameters.

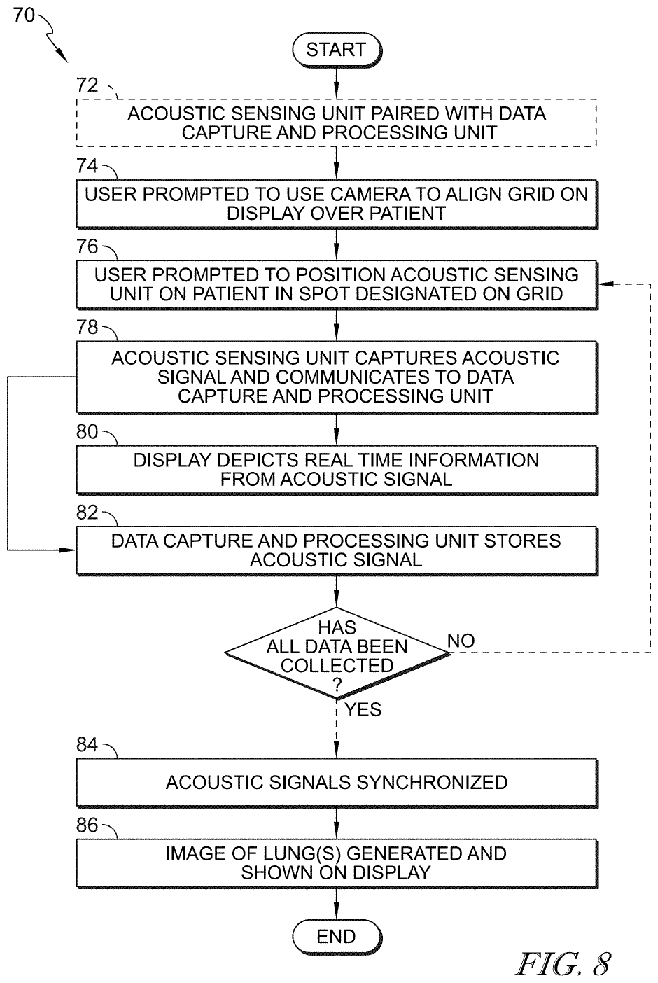

FIG. 8 depicts a process 70 of capturing and converting the at least one acoustic signal to an image 58, 64, 66 representing the patient's lungs with the image based lung auscultation system 10. At step 72, the acoustic sensing unit 12 is paired with the data capture and processing unit 14. As described above, step 72 may be achieved with wireless or wired connection(s). At step 74, the user interface 34 prompts the user 18 to use the camera 32 to align the grid 52 on the display 40 over the patient 16. At step 76, the user interface 34 prompts the user 18 to position the acoustic sensing unit 12 on the patient 16 at the designated location 54 on the grid 52. Once positioned, the acoustic sensing unit 12 captures the at least one acoustic signal and communicates the at least one acoustic signal to the data capture and processing unit 14 as described above. At step 80, the display 40 depicts real time information 55, 56, 57 from the at least one acoustic signal as described above. Simultaneously, at step 82 and as described above, the data capture and processing unit 14 stores the at least one acoustic signal. If all data has not been collected, then steps 76 to 82 are repeated. For example, steps 76 to 82 may be repeated for one respiratory cycle or more than one respiratory cycles. If all data has been collected, then the data capture and processing unit 14 synchronizes the at least one acoustic signal as described above at step 84. Finally, at step 86, the data capture and processing unit 14 generates an image 58, 64, 66 of the patient's lungs and shows the image 58, 64, 66 on the display 40, as described above.

Although this disclosure refers to specific embodiments, it will be understood by those skilled in the art that various changes in form and detail may be made without departing from the subject matter set forth in the accompanying claims.

1. An image based lung auscultation system comprising
an acoustic sensing unit positionable on a patient and having a controller and an acoustic sensor to capture and communicate an acoustic signal from a respiratory cycle of the patient, and
a data capture and processing unit including:
a camera operable by a user to identify a target position of the acoustic sensing unit on the patient,
a user interface including user inputs to operate the data capture and processing unit, a display, and a processor and a memory device storing instructions that, when executed by the processor, receive the user inputs, display an image generated by the camera on the display, display real-time information of the acoustic signal on the display, and display an output representing the patient's lungs on the display, and
a controller including a processor and a memory device storing instructions that, when executed by the processor, receive and store the acoustic signal from the acoustic sensing unit, generate real-time information of the acoustic signal, generate the output of the patient's lung(s), and communicate the real-time information and the output to the user interface.

2. The system of claim 1, wherein the acoustic signal includes more than one acoustic signal.

3. The system of claim 2, wherein the memory device of the controller further stores instructions that, when executed by the processor, synchronizes the more than one acoustic signal.

4. The system of claim 1, wherein the acoustic sensing unit is an electronic stethoscope.

5. The system of claim 1, wherein the acoustic sensing unit includes an acoustic sensor and a controller, wherein the controller includes a processor and a memory device storing instructions that, when executed by the processor, cause the acoustic sensor to capture the acoustic signal of the patient and communicate the acoustic signal to the controller of the data capture and processing unit.

6. The system of claim 1, wherein the data capture and processing unit is a mobile device.

7. The system of claim 1, wherein the memory device of the user interface further includes instructions, that, when executed by the processor, displays a guide on the display.

8. The system of claim 1, wherein the output representing the patient's lungs is an image.

9. The system of claim 1, wherein the acoustic sensing unit and the data capture and processing unit are wirelessly connected.

10. A method of capturing and converting an acoustic signal into an output representing a patient's lungs comprising the steps of:
prompting a user to position the acoustic sensing unit on the patient,
capturing the acoustic signal from a respiratory cycle of the patient with the acoustic sensing unit,
communicating the acoustic signal from the acoustic sensing unit to the data capture and processing unit,
storing the acoustic signal in the data capture and processing unit, and,
generating the output representing the patient's lungs.

11. The method of claim 10, further comprising the step of prompting the user to use a camera to position the acoustic sensing unit on the patient.

12. The method of claim 10, further comprising the step of depicting real-time information of the acoustic signal on a display of the data capture and processing unit.

13. The method of claim 10, further comprising the step of depicting the output representing the patient's lungs on a display of the data capture and processing unit.

14. The method of claim 10, wherein the acoustic signal includes more than one acoustic signal.

15. The method of claim 14, further comprising the step of synchronizing the more than one acoustic signals.

16. The method of claim 10, wherein the acoustic sensing unit is an electronic stethoscope.

17. The method of claim 10, wherein the acoustic sensing unit includes an acoustic sensor and a controller, wherein the controller includes a processor and a memory device storing instructions that, when executed by the processor, cause the acoustic sensor to capture the acoustic signal of the patient and communicate the acoustic signal to a controller of the data capture and processing unit.

18. The method of claim 10, wherein the data capture and processing unit is a mobile device.

19. The method of claim 10, wherein the output representing the patient's lungs is an image.

20. The method of claim 10, wherein the acoustic sensing unit and the data capture and processing unit are wirelessly connected.
